(19) United States
(12) Patent Application Publication
(10) Pub. No.: US 2022/0287608 A1
(43) Pub. Date: Sep. 15, 2022

(54) INATTENTIVE STATE DETERMINATION DEVICE

(71) Applicant: Panasonic Intellectual Property Management Co., Ltd., Osaka (JP)

(72) Inventors: Mika SUNAGAWA, Osaka (JP); Shinichi SHIKII, Nara (JP); Yoshihiro KOJIMA, Hyogo (JP); Daisuke WAKUDA, Kyoto (JP); Koki TAKAHASHI, Osaka (JP)

(21) Appl. No.: 17/428,246

(22) PCT Filed: Feb. 3, 2020
(86) PCT No.: PCT/JP2020/003865

§ 371 Date: Aug. 3, 2021

(30) Foreign Application Priority Data
Mar. 28, 2019 (JP) 2019-065141

(51) Int. Cl. A61B 5/16; A61B 5/00

(52) U.S. Cl. CPC A61B 5/168; A61B 5/7275; A61B 2560/0242; A61B 2503/22

(57) ABSTRACT

An inattentive state determination device includes: a drowsiness predictor that derives drowsiness prediction data for predicting an occurrence of drowsiness in a person; a drowsiness and inattentiveness deriver that derives a degree of drowsiness and inattentiveness of the person; and an inattentiveness determiner that determines whether the person is in an inattentive state, based on the drowsiness prediction data and a current degree of drowsiness and inattentiveness.

### CROSS-REFERENCE OF RELATED APPLICATIONS

This application is the U.S. National Phase under 35 U.S.C. § 371 of International Patent Application No. PCT/JP2020/003865, filed on Feb. 3, 2020, which in turn claims the benefit of Japanese Application No. 2019-065141, filed on Mar. 28, 2019, the entire disclosures of which Applications are incorporated by reference herein.

### TECHNICAL FIELD

The present disclosure relates to an inattentive state determination device that determines whether a person is in an inattentive state.

### BACKGROUND ART

Patent Literature (PTL) 1 discloses a device that detects a gaze of a driver and determines whether the driver is driving inattentively.

### CITATION LIST

### Patent Literature

[PTL 1] Japanese Unexamined Patent Application Publication No. 8-178712

### SUMMARY OF INVENTION

### Technical Problem

However, it is difficult to determine accurately whether a person is inattentive (hereinafter also referred to as “inattentive state”) by merely detecting a gaze of a driver as in the device disclosed by PTL 1.

In view of the above, the present disclosure provides an inattentive state determination device that is capable of determining accurately whether a person is in an inattentive state.

### Solution to Problem

An inattentive state determination device according to one aspect of the present disclosure includes: a drowsiness predictor that derives drowsiness prediction data for predicting an occurrence of drowsiness in a person; and an inattentiveness determiner that determines whether the person is in an inattentive state, based on the drowsiness prediction data.

An inattentive state determination device according to one aspect of the present disclosure includes: a drowsiness and inattentiveness deriver that derives a degree of drowsiness and inattentiveness indicating a degree of drowsiness and a degree of inattentiveness of a person; and an inattentiveness determiner that determines whether the person is in an inattentive state, based on a change in the degree of drowsiness and inattentiveness.

An inattentive state determination device according to one aspect of the present disclosure includes: a fatigue predictor that derives fatigue prediction data for predicting an occurrence of fatigue in a person; and an inattentiveness determiner that determines whether the person is in an inattentive state, based on the fatigue prediction data.

An inattentive state determination device according to one aspect of the present disclosure includes: a fatigue and inattentiveness deriver that derives a degree of fatigue and inattentiveness indicating a degree of fatigue and a degree of inattentiveness of a person; and an inattentiveness determiner that determines whether the person is in an inattentive state, based on a change in the degree of fatigue and inattentiveness.

### Advantageous Effects of Invention

According to the above-described aspects, it is possible to determine accurately whether a person is in an inattentive state.

### BRIEF DESCRIPTION OF DRAWINGS

FIG. 1A is a schematic diagram illustrating one example of an inattentive state determination system including an inattentive state determination device according to Embodiment 1.

FIG. 1B is a schematic diagram illustrating another example of the inattentive state determination system including the inattentive state determination device according to Embodiment 1.

FIG. 2 is a block diagram illustrating a functional configuration of the inattentive state determination device according to Embodiment 1.

FIG. 3 is a diagram illustrating an example of determining an inattentive state using the inattentive state determination device according to Embodiment 1.

FIG. 4 is a flow chart illustrating operations of the inattentive state determination device according to Embodiment 1.

FIG. 5 is a block diagram illustrating a functional configuration of an inattentive state determination device according to Embodiment 2.

FIG. 6 is a diagram illustrating an example of determining an inattentive state using the inattentive state determination device according to Embodiment 2.

FIG. 7 is a flow chart illustrating operations of the inattentive state determination device according to Embodiment 2.

FIG. 8 is a block diagram illustrating a functional configuration of an inattentive state determination device according to Embodiment 3.

FIG. 9 is a diagram illustrating an example of determining an inattentive state using the inattentive state determination device according to Embodiment 3.

FIG. 10 is a flow chart illustrating operations of the inattentive state determination device according to Embodiment 3.

FIG. 11 is a block diagram illustrating a functional configuration of an inattentive state determination device according to Embodiment 4.

FIG. 12 is a block diagram illustrating a functional configuration of an inattentive state determination device according to Embodiment 5.

FIG. 13 is a diagram illustrating an example of determining an inattentive state using the inattentive state determination device according to Embodiment 5.

FIG. 14 is a block diagram illustrating a functional configuration of an inattentive state determination device according to Embodiment 6.

FIG. 15 is a diagram illustrating an example of determining an inattentive state using the inattentive state determination device according to Embodiment 6.

FIG. 16 is a block diagram illustrating a functional configuration of an inattentive state determination device according to Embodiment 7.

FIG. 17 is a diagram illustrating an example of determining an inattentive state using the inattentive state determination device according to Embodiment 7.

### DESCRIPTION OF EMBODIMENTS

For example, when a person is driving a vehicle or working in a room, the person may be in an inattentive state. The inattentive state refers to a state in which a person is not paying attention to something to which the person should pay attention in a current situation, or especially a state in which a person is thinking about something different from a thing to which the person should pay attention. When a person is in the inattentive state, the person, for example, keeps looking forward inattentively without moving the face or fixes a gaze unconsciously. There is a drowsiness occurrence state as a state similar to this inattentive state. When a person is in the drowsiness occurrence state, the person keeps looking forward without moving the face or fixes a gaze unconsciously.

When a person is in the inattentive state or the drowsiness occurrence state, the person may drive a vehicle or work indoors slowly or carelessly. For this reason, when a person is in the inattentive state or the drowsiness occurrence state, it is desirable to bring the person back to a normal state.

For example, when a person is in the inattentive state, it is possible to bring the person back to the normal state immediately by calling the person's attention. On the other hand, when a person is in the drowsiness occurrence state, the person's drowsiness cannot be shaken off by merely calling the person's attention, and it is necessary to apply a strong stimulus such as splashing cold water on the person. However, applying a strong stimulus to a person in the inattentive state may cause the person to act abnormally in surprise. In this way, since a means to bring back to the normal state differs between the inattentive state and the drowsiness occurrence state, it is necessary to detect whether a person is in the inattentive state or the drowsiness occurrence state before the person is brought back to the normal state.

As stated above, however, since the inattentive state and the drowsiness occurrence state both cause a person to keep looking forward without moving the face or fix a gaze unconsciously, it is difficult to determine whether the person is in the inattentive state by merely checking the appearance of the person.

In view of the above, since an inattentive state determination device according to the present embodiment has the following configurations, the inattentive state determination device is capable of determining accurately whether a person is in the inattentive state.

Stated differently, an inattentive state determination device according to one aspect of the present disclosure includes: a drowsiness predictor that derives drowsiness prediction data for predicting an occurrence of drowsiness in a person; and an inattentiveness determiner that determines whether the person is in an inattentive state, based on the drowsiness prediction data.

As stated above, determining an inattentive state based on drowsiness prediction data makes it possible to distinguish between the inattentive state and the drowsiness occurrence state. Accordingly, it is possible to determine accurately whether a person is in the inattentive state.

Moreover, the inattentive state determination device may further include a drowsiness and inattentiveness deriver that derives a degree of drowsiness and inattentiveness indicating a degree of drowsiness and a degree of inattentiveness of the person, and the the inattentiveness determiner may determine whether the person is in the inattentive state, based on the drowsiness prediction data and a current degree of drowsiness and inattentiveness.

As stated above, determining an inattentive state based on drowsiness prediction data and a degree of drowsiness and inattentiveness makes it possible to distinguish between the inattentive state and the drowsiness occurrence state. Accordingly, it is possible to determine accurately whether a person is in the inattentive state.

Moreover, the inattentive state determination device may further include: a user information obtainer that obtains information about the person; and an environmental information obtainer that obtains information about an environment surrounding the person, and the drowsiness predictor may derive the drowsiness prediction data, based on the information obtained by the user information obtainer and the information obtained by the environmental information obtainer.

With this configuration, since it is possible to derive drowsiness prediction data accurately, it is possible to distinguish the inattentive state and the drowsiness occurrence state accurately. Accordingly, it is possible to determine accurately whether a person is in the inattentive state.

Moreover, the inattentive state determination device may further include: a user information obtainer that obtains information about the person; and an environmental information obtainer that obtains information about an environment surrounding the person, and the drowsiness and inattentiveness deriver may derive the degree of drowsiness and inattentiveness, based on the information obtained by the user information obtainer and the information obtained by the environmental information obtainer.

With this configuration, since it is possible to derive a degree of drowsiness and inattentiveness accurately, it is possible to distinguish the inattentive state and the drowsiness occurrence state accurately. Accordingly, it is possible to determine accurately whether a person is in the inattentive state.

Moreover, the inattentive state determination device may further include: a user information obtainer that obtains information about the person; and an environmental information obtainer that obtains information about an environment surrounding the person, and the inattentiveness determiner may determine a likelihood of the inattentive state, based on the information obtained by the user information obtainer and the information obtained by the environmental information obtainer, and may further determine whether the person is in the inattentive state, based on the likelihood of the inattentive state.

As stated above, determining a likelihood of an inattentive state makes it possible to distinguish between the inattentive state and the drowsiness occurrence state accurately. Accordingly, it is possible to determine accurately whether a person is in the inattentive state.

An inattentive state determination device according to one aspect of the present disclosure includes: a drowsiness and inattentiveness deriver that derives a degree of drowsiness and inattentiveness indicating a degree of drowsiness and a degree of inattentiveness of a person; and an inattentiveness determiner that determines whether the person is in an inattentive state, based on a change in the degree of drowsiness and inattentiveness.

As stated above, determining an inattentive state based on a change in a degree of drowsiness and inattentiveness makes it possible to distinguish between the inattentive state and the drowsiness occurrence state. Accordingly, it is possible to determine accurately whether a person is in the inattentive state.

Moreover, the inattentive state determination device may further include a history holder that holds a drowsiness and inattentiveness history that is a history of degrees of drowsiness and inattentiveness, and the inattentiveness determiner may compare the drowsiness and inattentiveness history and a current degree of drowsiness and inattentiveness to determine whether the person is in the inattentive state.

As stated above, comparing a drowsiness and inattentiveness history and a degree of drowsiness and inattentiveness to determine an inattentive state makes it possible to distinguish between the inattentive state and the drowsiness occurrence state. Accordingly, it is possible to determine accurately whether a person is in the inattentive state.

Moreover, the inattentive state determination device may further include: a user information obtainer that obtains information about the person; and an environmental information obtainer that obtains information about an environment surrounding the person, and the drowsiness and inattentiveness deriver may derive the degree of drowsiness and inattentiveness, based on the information obtained by the user information obtainer and the information obtained by the environmental information obtainer.

With this configuration, since it is possible to derive a degree of drowsiness and inattentiveness accurately, it is possible to distinguish the inattentive state and the drowsiness occurrence state accurately. Accordingly, it is possible to determine accurately whether a person is in the inattentive state. Moreover, the inattentiveness determiner may determine a likelihood of the inattentive state, based on the information obtained by the user information obtainer and the information obtained by the environmental information obtainer, and may further determine whether the person is in the inattentive state, based on the likelihood of the inattentive state.

As stated above, determining a likelihood of an inattentive state makes it possible to distinguish between the inattentive state and the drowsiness occurrence state accurately. Accordingly, it is possible to determine accurately whether a person is in the inattentive state.

It should be noted that the relationships between the inattentive state and the drowsiness occurrence state have been described above, but the present disclosure is not limited to those examples. The same applies to the relationships between the inattentive state and a fatigue occurrence state.

To put it another way, an inattentive state determination device according to one aspect of the present disclosure includes: a fatigue predictor that derives fatigue prediction data for predicting an occurrence of fatigue in a person; and an inattentiveness determiner that determines whether the person is in an inattentive state, based on the fatigue prediction data.

As stated above, determining an inattentive state based on fatigue prediction data makes it possible to distinguish between the inattentive state and the fatigue occurrence state. Accordingly, it is possible to determine accurately whether a person is in the inattentive state.

An inattentive state determination device according to one aspect of the present disclosure includes: a fatigue and inattentiveness deriver that derives a degree of fatigue and inattentiveness indicating a degree of fatigue and a degree of inattentiveness of a person; and an inattentiveness determiner that determines whether the person is in an inattentive state, based on a change in the degree of fatigue and inattentiveness.

As stated above, determining an inattentive state based on a change in a degree of fatigue and inattentiveness makes it possible to distinguish between the inattentive state and the fatigue occurrence state. Accordingly, it is possible to determine accurately whether a person is in the inattentive state.

It should be noted that these general or specific aspects may be realized by a system, a method, an integrated circuit, a computer program, or a computer-readable recording medium such as CD-ROM, or may be realized by any combination of systems, methods, integrated circuits, computer programs, and recording media.

Hereinafter, an inattentive state determination device according to one aspect of the present disclosure will be described in detail with reference to the drawings.

It should be noted that each of the exemplary embodiments described below shows a specific example of the present disclosure. The numerical values, shapes, materials, constituent elements, the arrangement and connection of the constituent elements, steps, the order of the steps, etc. indicated the following exemplary embodiments are mere examples, and are not intended to limit the scope of the present disclosure. Moreover, among the constituent elements in the following exemplary embodiments, those not recited in any one of the independent claims indicating the broadest concepts are described as optional constituent elements.

### Embodiment 1

### [1-1. Configuration of Inattentive State Determination System]

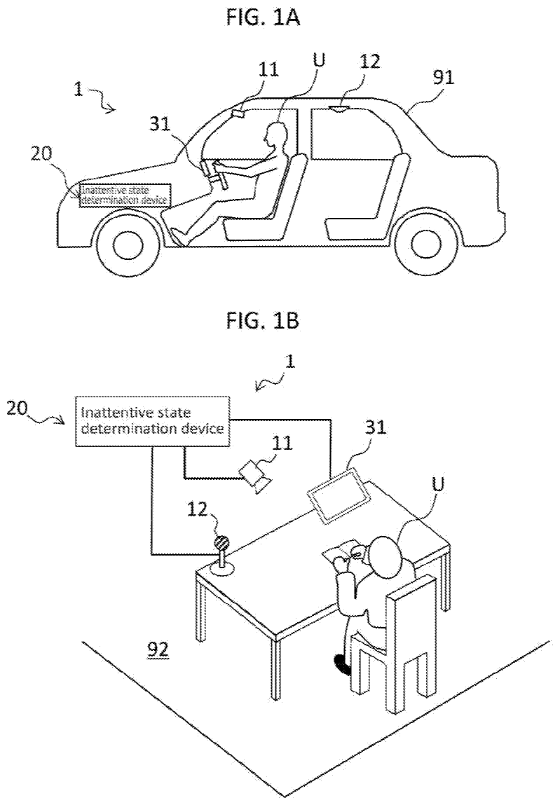

First, the following describes a configuration of inattentive state determination system 1 including inattentive state determination device 20 according to Embodiment 1 with reference to FIG. 1A and FIG. 1B.

FIG. 1A is a schematic diagram illustrating one example of inattentive state determination system 1 including inattentive state determination device 20 according to Embodiment 1. FIG. 1A shows an example in which inattentive state determination system 1 is provided to vehicle 91.

FIG. 1B is a schematic diagram illustrating another example of inattentive state determination system 1 including inattentive state determination device 20. FIG. 1B shows an example in which inattentive state determination system 1 is provided to room 92.

As shown by FIG. 1A and FIG. 1B, inattentive state determination system 1 is provided in a predetermined space of vehicle 91 or room 92, for example.

Inattentive state determination system 1 includes inattentive state determination device 20, first inputter 11, second inputter 12, and notifier 31.

Inattentive state determination device 20 determines whether a person is in an inattentive state. Inattentive state determination device 20 includes, for example, a micro processing unit and memory. First inputter 11, second inputter 12, and notifier 31 are connected to inattentive state determination device 20. Inattentive state determination device 20 will be described in detail later.

First inputter 11 is a device for inputting to inattentive state determination device 20 information about a person using inattentive state determination device 20, that is, user U.

First inputter 11 is, for example, a camera, time-of-flight (ToF), a balance sensor, or a biological sensor. Information such as a gaze, a pupil diameter, an eye-opening state, blinking, a head movement, a body movement frequency, a body temperature, thermal sensation, a heart rate, breathing, and breath components of user U is inputted as information about user U to first inputter 11. Moreover, information such as a dinning history, an action history, a schedule, a wake-up time, a sleep time of user U may be inputted to first inputter 11. It should be noted that when inattentive state determination device 20 is provided to vehicle 91, information such as steering, accelerating, and a distance between vehicles may be inputted as information about user U. Moreover, when inattentive state determination device 20 is provided to room 92, information such as an user operation on a personal computer may be inputted as information about user U.

Second inputter 12 is a device for inputting to inattentive state determination device 20 information about an environment surrounding user U.

Second inputter 12 is one of various types of environmental sensors. Information such as an illuminance, a color, a temperature, a wind speed, ambient sound, smell, and air components is inputted as information about an environment to second inputter 12. It should be noted that when inattentive state determination device 20 is provided to vehicle 91, information such as a passenger, vehicles in front and behind, a traveling route, traffic jam information, and a road environment may be inputted as information about an environment. Moreover, when inattentive state determination device 20 is provided to room 92, information such as browse information recorded on a personal computer and information about other people in the same vicinity may be inputted as information about an environment.

Although the example in which the information about user U is inputted to first inputter 11 and the information about the environment is inputted to second inputter 12 has been described above, the present embodiment is not limited to the example. For example, information about an environment may be inputted using a camera etc. of first inputter 11, or information about user U may be inputted using a sensor etc. of second inputter 12.

Notifier 31 is a device that notifies that a person is in an inattentive state, and is an image display or a speaker, for example. Moreover, notifier 31 may be a smartphone or an information terminal such as a tablet terminal, a stimulating device that provides stimulation such as a vibration to a seat in which a person sits, or an in-vehicle head-up display.

### [1-2. Configuration of Inattentive State Determination Device]

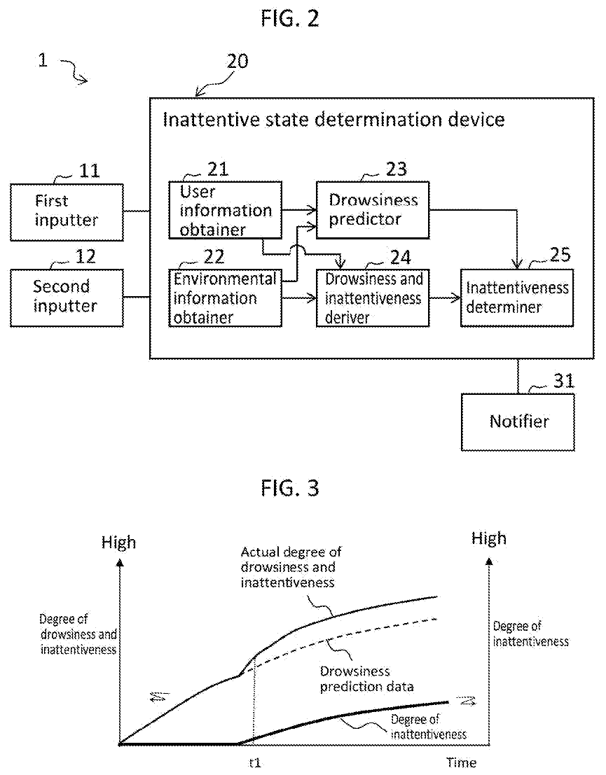

Next, the following describes a configuration of inattentive state determination device 20 with reference to FIG. 2 and FIG. 3.

FIG. 2 is a block diagram illustrating a functional configuration of inattentive state determination device 20. It should be noted that FIG. 2 also shows first inputter 11, second inputter 12, and notifier 31 connected to inattentive state determination device 20.

As shown by FIG. 2, inattentive state determination device 20 includes user information obtainer 21, environmental information obtainer 22, drowsiness predictor 23, drowsiness and inattentiveness deriver 24, and inattentiveness determiner 25.

User information obtainer 21 obtains information about user U inputted by first inputter 11 and second inputter 12. The information obtained by user information obtainer 21 is outputted to drowsiness predictor 23 and drowsiness and inattentiveness deriver 24.

Environmental information obtainer 22 obtains information about an environment inputted by first inputter 11 and second inputter 12. The information obtained by environmental information obtainer 22 is outputted to drowsiness predictor 23 and drowsiness and inattentiveness deriver 24.

Drowsiness predictor 23 predicts the occurrence of drowsiness in a person. Specifically, drowsiness predictor 23 derives drowsiness prediction data for predicting the occurrence of drowsiness in user U, based on the information about user U obtained by user information obtainer 21 and the information about the environment obtained by environmental information obtainer 22.

Drowsiness prediction data is a function for predicting the future occurrence of drowsiness in user U. Drowsiness prediction data can be derived by, for example, conducting multivariate analysis on data about degrees of drowsiness in user U for the past 10 minutes when user U is not in an inattentive state. It should be noted that a degree of drowsiness is a value indicating a progress of drowsiness. The degree of drowsiness is represented by, for example, a five-point scale, and the value increases with an increase in the degree of drowsiness. For example, a degree of drowsiness is 1 when a person's blinking is periodically stable, 3 when the person's blinking is slow, and 5 when the person's eyes are closed.

Moreover, drowsiness predictor 23 predicts the occurrence of drowsiness, based on information about an action of user U such as dining, exercising, sleeping, or working. Specifically, drowsiness predictor 23 predicts that drowsiness will likely fall upon user U after user U eats a meal, after user U exercises, when user U has lack of sleep, or when user U is reading documents. Furthermore, drowsiness predictor 23 predicts the occurrence of drowsiness, based on information about an environment surrounding user U such as an illuminance, a temperature, an environmental color, a CO₂ concentration, and noise. Specifically, drowsiness predictor 23 predicts that drowsiness will likely fall upon user U when the surrounding environment is dark, when the surrounding environment is warm, when the surrounding environment has a warm color, when the surrounding environment has a high CO₂ concentration, or when the surrounding environment is quiet.

The drowsiness prediction data obtained by drowsiness predictor 23 is outputted to inattentiveness determiner 25.

Drowsiness and inattentiveness deriver 24 derives a degree of drowsiness and inattentiveness indicating a degree of drowsiness and a degree of inattentiveness of a person. Specifically, drowsiness and inattentiveness deriver 24 derives a current degree of drowsiness and inattentiveness of user U, based on the information about user U obtained by user information obtainer 21 and the information about the environment obtained by environmental information obtainer 22.

As stated above, it is difficult to determine whether a person is in the drowsiness occurrence state or the inattentive state by merely checking the appearance of the person. For this reason, the degree of drowsiness and inattentiveness obtained based on the information about user U and the information about the environment is a value including both the degree of drowsiness and the degree of inattentiveness. A degree of drowsiness and inattentiveness is represented by, for example, a ten-point scale, and a value increases with an increase in a degree of drowsiness and inattentiveness. For example, a degree of drowsiness and inattentiveness is 1 when a gaze of a person is shifted frequently, 5 when the gaze is shifted slowly, and 10 when the gaze is completely fixed.

Drowsiness and inattentiveness deriver 24 derives a degree of drowsiness and inattentiveness, based on, for example, a gaze of a person, the pupils of the person, the closed eyes of the person, a head swing by the person, and a body movement frequency of the person. Specifically, drowsiness and inattentiveness deriver 24 determines a degree of drowsiness and inattentiveness to be high when the gaze is stagnant, the pupil diameter is changing, the eyes are closed, the head is swinging, or the body movement is reduced. Moreover, drowsiness and inattentiveness deriver 24 may determine a degree of drowsiness and inattentiveness to be high when, for example, a steering frequency is reduced or a distance between vehicles is not constant and zigzag driving is performed in the case where vehicle 91 is driven.

The degree of drowsiness and inattentiveness derived by drowsiness and inattentiveness deriver 24 is outputted to inattentiveness determiner 25.

Inattentiveness determiner 25 determines whether a person is in an inattentive state. Specifically, inattentiveness determiner 25 derives a degree of inattentiveness of the person, based on the drowsiness prediction data derived by drowsiness predictor 23 and the degree of drowsiness and inattentiveness obtained by drowsiness and inattentiveness deriver 24, and determines whether the person is in the inattentive state. The following describes how inattentiveness determiner 25 determines an inattentive state with reference to FIG. 3.

FIG. 3 is a diagram illustrating an example of determining an inattentive state using inattentive state determination device 20.

The horizontal axis, left vertical axis, and right vertical axis of FIG. 3 indicate a time for monitoring user U, a degree of drowsiness and inattentiveness, and a degree of inattentiveness, respectively. In FIG. 3, an actual degree of drowsiness and inattentiveness obtained by drowsiness and inattentiveness deriver 24 is shown by the solid line, and drowsiness prediction data derived by drowsiness predictor 23 is shown by the broken line.

As shown by FIG. 3, since drowsiness gradually progresses with time, drowsiness prediction data is shown by a gentle upward curve. In contrast, an inattentive state occurs depending on user U and appears as a sudden change in the middle of monitoring user U. For this reason, when an inattentive state occurs, a curve showing an actual degree of drowsiness and inattentiveness considerably deviates from a curve showing drowsiness prediction data.

Using these characteristics, it is possible to represent a degree of inattentiveness with a difference (see the thick solid line in FIG. 3) between an actual degree of drowsiness and inattentiveness and drowsiness prediction data. For example, the degree of inattentiveness at time t1 is a value obtained by subtracting a value of the drowsiness prediction data at time t1 from a value of the actual degree of drowsiness and inattentiveness at time t1. A predetermined threshold value may be set for a degree of inattentiveness in order to determine whether user U is in the inattentive state. When the degree of inattentiveness derived above is greater than or equal to the predetermined threshold value, user U may be determined to be in the inattentive state; and when the degree of inattentiveness derived above is less than the predetermined threshold value, user U may be determined to be not in the inattentive state. In this manner, it is possible to determine whether user U is in the inattentive state at time t1.

It should be noted that in the case where a degree of drowsiness and inattentiveness and a degree of inattentiveness have a mutually different evaluation scale, it is desirable to, when a degree of inattentiveness is derived, make the evaluation scales conform to one another by performing predetermined weighting on a difference between an actual degree of drowsiness and inattentiveness and drowsiness prediction data.

Inattentive state determination device 20 according to the present embodiment includes: drowsiness predictor 23 that derives drowsiness prediction data for predicting the occurrence of drowsiness in a person; drowsiness and inattentiveness deriver 24 that derives a degree of drowsiness and inattentiveness of the person; and inattentiveness determiner 25 that determines whether the person is in an inattentive state, based on the drowsiness prediction data and a current degree of drowsiness and inattentiveness.

As stated above, determining an inattentive state based on drowsiness prediction data and a degree of drowsiness and inattentiveness makes it possible to distinguish between the inattentive state and the drowsiness occurrence state. Accordingly, it is possible to determine accurately whether a person is in the inattentive state.

### [1-3. Operations of Inattentive State Determination Device]

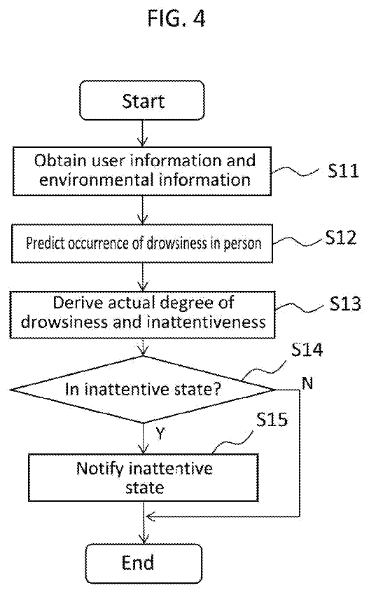

Next, the following describes operations of inattentive state determination device 20 with reference to FIG. 4.

FIG. 4 is a flow chart illustrating the operations of inattentive state determination device 20.

First, inattentive state determination device 20 obtains user information and environmental information (step S11). Specifically, user information obtainer 21 obtains the user information, and environmental information obtainer 22 obtains the environmental information. It should be noted that user information is inputted mainly by first inputter 11, and environmental information is inputted mainly by second inputter 12.

Next, inattentive state determination device 20 predicts the occurrence of drowsiness in a person (step S12). Specifically, drowsiness predictor 23 derives drowsiness prediction data, based on the user information and the environmental information.

Then, inattentive state determination device 20 derives a current degree of drowsiness and inattentiveness of the person (step S13). Specifically, drowsiness and inattentiveness deriver 24 derives a degree of drowsiness and inattentiveness indicating a degree of drowsiness and a degree of inattentiveness, based on the user information and the environmental information.

After that, inattentive state determination device 20 determines whether the person is in an inattentive state (step S14). Specifically, inattentiveness determiner 25 derives a degree of inattentiveness, based on drowsiness prediction data and an actual degree of drowsiness and inattentiveness, and determines whether the person is in the inattentive state.

Finally, when inattentiveness determiner 25 determines that the person is in the inattentive state (Y in S14), inattentiveness determiner 25 causes notifier 31 to notify that the person is in the inattentive state (step S15). On the other hand, when inattentiveness determiner 25 determines that the person is not in the inattentive state (N in S14), the process is ended without providing an output to notifier 31. Those steps S11 to S15 make it possible to determine whether the person is in the inattentive state. For example, steps S11 to 1515 may be performed repeatedly in vehicle 91 or room 92.

As stated above, an inattentive state determination method to be performed using inattentive state determination device 20 includes: deriving drowsiness prediction data for predicting the occurrence of drowsiness in a person; deriving a degree of drowsiness and inattentiveness of the person; and determining whether the person is in an inattentive state, based on the drowsiness prediction data and a current degree of drowsiness and inattentiveness. This method makes it possible to determine accurately whether the person is in the inattentive state.

### Embodiment 2

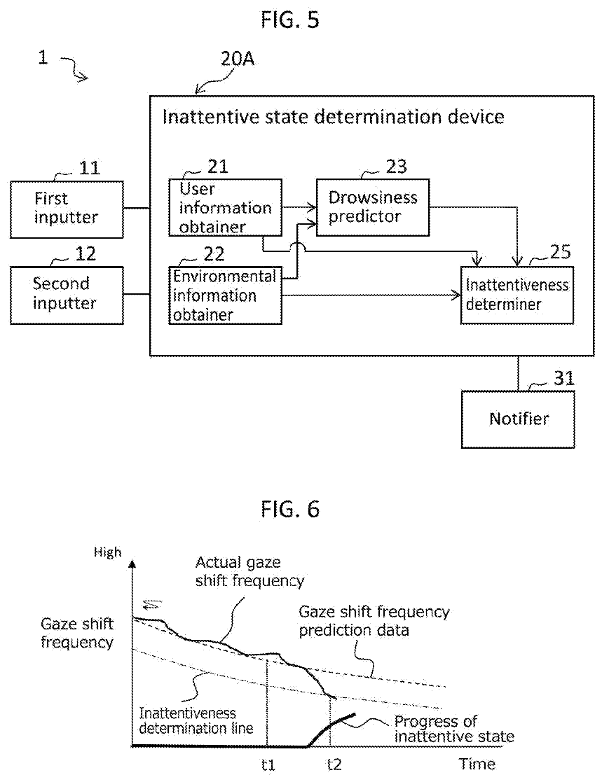

Next, the following describes inattentive state determination device 20A according to Embodment 2 with reference to FIG. 5 to FIG. 7. Embodment 2 describes an example of determining an inattentive state using prediction data obtained by drowsiness predictor 23 but not using a degree of drowsiness and inattentiveness.

FIG. 5 is a block diagram illustrating a functional configuration of inattentive state determination device 20A according to

Embodment 2. FIG. 6 is a diagram illustrating an example of determining an inattentive state using inattentive state determination device 20A.

As shown by FIG. 5, inattentive state determination device 20A includes user information obtainer 21, environmental information obtainer 22, drowsiness predictor 23, and inattentiveness determiner 25. Inattentive state determination device 20A according to Embodment 2 does not include drowsiness and inattentiveness deriver 24.

Drowsiness predictor 23 predicts the occurrence of drowsiness in a person. Specifically, drowsiness predictor 23 derives gaze shift frequency prediction data (see FIG. 6) related to drowsiness prediction data, based on information about user U obtained by user information obtainer 21 and information about an environment obtained by environmental information obtainer 22. A gaze shift frequency is the number of times a gaze is shifted during a predetermined period. A lower gaze shift frequency indicates a higher degree of drowsiness.

Gaze shift frequency prediction data is a function for predicting a future gaze shift frequency of user U. Gaze shift frequency prediction data is derived based on, for example, the above-described drowsiness prediction data. Moreover, drowsiness predictor 23 derives an inattentiveness determination line (see FIG. 6) that is a threshold value for determining whether a person is in an inattentive state. An inattentiveness determination line is provided along gaze shift frequency prediction data and at predetermined intervals with respect to the gaze shift frequency prediction data. It should be noted that the inattentiveness determination line may be provided such that the predetermined intervals vary according to a value of the gaze shift frequency prediction data. The gaze shift frequency prediction data and the inattentiveness determination line obtained by drowsiness predictor 23 are outputted to inattentiveness determiner 25.

It should be noted that although the gaze shift frequency prediction data has been shown as data related to the drowsiness prediction data in the above, the present embodiment is not limited to this example. For example, data related to drowsiness prediction data may be driving accuracy prediction data or working accuracy prediction data.

Inattentiveness determiner 25 determines whether a person is in an inattentive state. Specifically, inattentiveness determiner 25 determines whether the person is in the inattentive state, based on the gaze shift frequency prediction data and the inattentiveness determination line derived by drowsiness predictor 23. The following describes how inattentiveness determiner 25 determines an inattentive state with reference to FIG. 6.

The horizontal axis and left vertical axis of FIG. 6 indicate a time for monitoring user U and a gaze shift frequency, respectively. In FIG. 6, an actual gaze shift frequency obtained by user information obtainer 21 is shown by the solid line, and gaze shift frequency prediction data derived by drowsiness predictor 23 is shown by the broken line.

As shown by FIG. 6, since drowsiness gradually progresses with time, gaze shift frequency prediction data is shown by a gentle downward curve. In contrast, an inattentive state occurs depending on user U and appears as a sudden change in the middle of monitoring user U. For this reason, when an inattentive state occurs, a curve showing an actual gaze shift frequency considerably deviates from a curve showing the gaze shift frequency prediction data.

FIG. 6 shows an example in which an inattentive state does not occur at time t1 but occurs at time t2. As shown by FIG. 6, in the present embodiment, when an actual gaze shift frequency considerably deviates from prediction data and crosses an inattentiveness determination line, an inattentive state is determined to have occurred.

Inattentive state determination device 20A according to Embodment 2 includes: drowsiness predictor 23 that derives drowsiness prediction data for predicting the occurrence of drowsiness in a person; and inattentiveness determiner 25 that determines whether the person is in an inattentive state, based on the drowsiness prediction data.

As stated above, determining an inattentive state based on drowsiness prediction data makes it possible to distinguish between the inattentive state and the drowsiness occurrence state. Accordingly, it is possible to determine accurately whether a person is in the inattentive state.

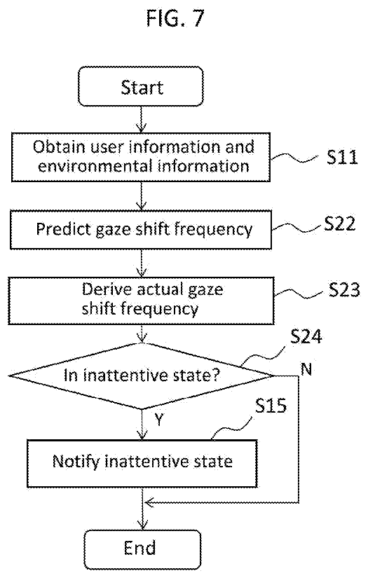

FIG. 7 is a flow chart illustrating operations of inattentive state determination device 20A.

First, inattentive state determination device 20A obtains user information and environmental information (step S11).

Next, inattentive state determination device 20A predicts a gaze shift frequency of a person (step S22). Specifically, drowsiness predictor 23 derives gaze shift frequency prediction data, based on drowsiness prediction data. Then, inattentive state determination device 20A derives a current gaze shift frequency of the person (step S23). Specifically, inattentive state determination device 20A derives the gaze shift frequency, based on user information obtained by user information obtainer 21.

After that, inattentive state determination device 20A determines whether the person is in an inattentive state (step S24). Specifically, inattentiveness determiner 25 determines whether the person is in the inattentive state, based on an actual gaze shift frequency, gaze shift frequency prediction data, and an inattentiveness determination line. The subsequent step is the same as Embodiment 1.

As stated above, an inattentive state determination method to be performed using inattentive state determination device 20A includes: deriving drowsiness prediction data for predicting the occurrence of drowsiness in a person; and determining whether the person is in an inattentive state, based on the drowsiness prediction data. This method makes it possible to determine accurately whether the person is in the inattentive state.

### Embodiment 3

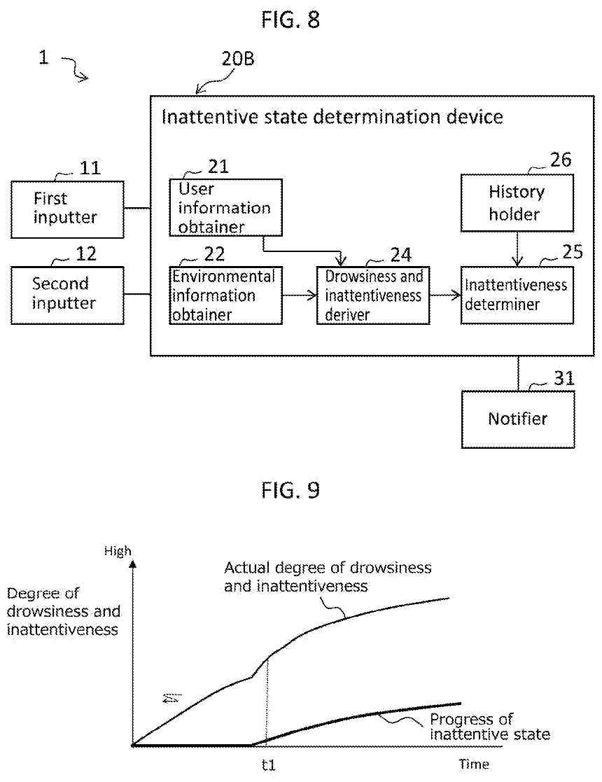

Next, the following describes inattentive state determination device 20B according to Embodiment 3 with reference to FIG. 8 to FIG. 10. Embodiment 3 describes an example of determining an inattentive state, based on a change in an actual degree of drowsiness and inattentiveness, without using drowsiness predictor 23.

FIG. 8 is a block diagram illustrating a functional configuration of inattentive state determination device 20B according to Embodiment 3. FIG. 9 is a diagram illustrating an example of determining an inattentive state using inattentive state determination device 20B.

As shown by FIG. 8, inattentive state determination device 20B includes user information obtainer 21, environmental information obtainer 22, drowsiness and inattentiveness deriver 24, and inattentiveness determiner 25. In addition, inattentive state determination device 20B includes history holder 26. Inattentive state determination device 20B according to Embodiment 3 does not include drowsiness predictor 23.

Drowsiness and inattentiveness deriver 24 derives a degree of drowsiness and inattentiveness indicating a degree of drowsiness and a degree of inattentiveness of a person. Specifically, drowsiness and inattentiveness deriver 24 derives a current degree of drowsiness and inattentiveness of user U, based on information about user U obtained by user information obtainer 21 and information about an environment obtained by environmental information obtainer 22.

The degree of drowsiness and inattentiveness derived by drowsiness and inattentiveness deriver 24 is outputted to inattentiveness determiner 25.

Inattentiveness determiner 25 determines whether a person is in an inattentive state. Specifically, inattentiveness determiner 25 determines whether the person is in the inattentive state, based on a change in the degree of drowsiness and inattentiveness obtained by drowsiness and inattentiveness deriver 24. The following describes how inattentiveness determiner 25 determines an inattentive state with reference to FIG. 9.

The horizontal axis and left vertical axis of FIG. 9 indicate a time for monitoring user U and a degree of drowsiness and inattentiveness, respectively. In FIG. 9, an actual degree of drowsiness and inattentiveness obtained by drowsiness and inattentiveness deriver 24 is shown by the solid line.

As shown by FIG. 9, since drowsiness gradually progresses with time, the drowsiness is shown by a gentle upward curve. In contrast, an inattentive state occurs depending on user U and appears as a sudden change in the middle of monitoring user U. For this reason, as shown by FIG. 9, when an inattentive state occurs, a curve showing a degree of drowsiness and inattentiveness changes suddenly with respect to a curve showing a past degree of drowsiness and inattentiveness. For example, differential values before and after a change in a degree of drowsiness and inattentiveness may be compared in order to determine whether user U is in the inattentive state. When a difference between the differential values is greater than or equal to a predetermined value, user U may be determined to be in the inattentive state; and when the difference between the differential values is less than the predetermined value, user U may be determined to be not in the inattentive state. In this manner, it is possible to determine whether user U is in the inattentive state.

Inattentive state determination device 20B according to the present embodiment includes: drowsiness and inattentiveness deriver 24 that derives a degree of drowsiness and inattentiveness indicating a degree of drowsiness and a degree of inattentiveness of a person;

and inattentiveness determiner 25 that determines whether the person is in an inattentive state, based on a change in the degree of drowsiness and inattentiveness.

As stated above, determining an inattentive state based on a change in a degree of drowsiness and inattentiveness makes it possible to distinguish between the inattentive state and the drowsiness occurrence state. Accordingly, it is possible to determine accurately whether a person is in the inattentive state.

It should be noted that inattentive state determination device 20B may include history holder 26 that holds a drowsiness and inattentiveness history that is a history of degrees of drowsiness and inattentiveness. In addition, inattentiveness determiner 25 may compare the drowsiness and inattentiveness history and a current degree of drowsiness and inattentiveness to determine whether the person is in the inattentive state. For example, history holder 26 may hold a drowsiness and inattentiveness history for a past few minutes while updating the drowsiness and inattentiveness history, and inattentiveness determiner 25 may determine that the person is in the inattentive state when a current degree of drowsiness and inattentiveness changes with at least a predetermined amplitude with respect to the drowsiness and inattentiveness history.

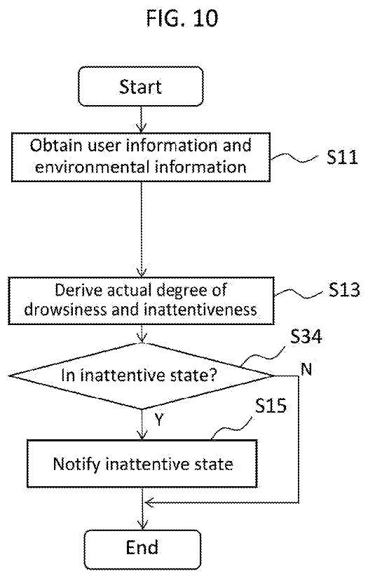

FIG. 10 is a flow chart illustrating operations of inattentive state determination device 20B.

First, inattentive state determination device 20B obtains user information and environmental information (step S11). Next, inattentive state determination device 20B derives a current degree of drowsiness and inattentiveness of a person (step S13).

Then, inattentive state determination device 20B determines whether the person is in an inattentive state (step S34). Specifically, inattentiveness determiner 25 determines whether the person is in the inattentive state according to whether a change in a degree of drowsiness and inattentiveness is sudden. The subsequent step is the same as Embodiment 1.

As stated above, an inattentive state determination method to be performed using inattentive state determination device 20B includes: deriving a degree of drowsiness and inattentiveness of a person; and determining whether the person is in an inattentive state, based on a change in the degree of drowsiness and inattentiveness. This method makes it possible to determine accurately whether the person is in the inattentive state.

### Embodiment 4

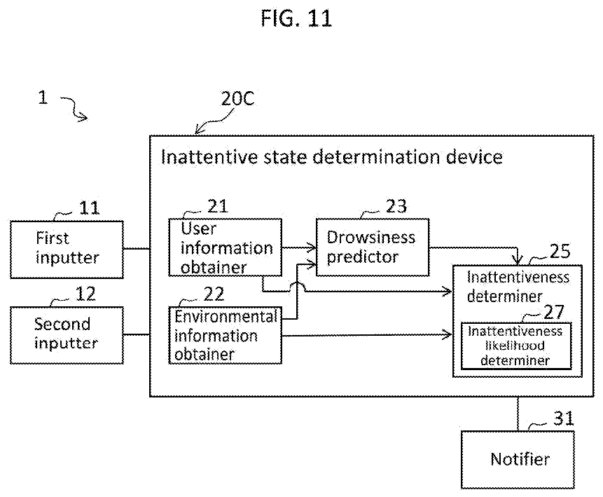

Next, the following describes inattentive state determination device 20C according to Embodiment 4 with reference to FIG. 11. Embodiment 4 describes an example in which inattentive state determination device 20C includes inattentiveness likelihood determiner 27.

FIG. 11 is a block diagram illustrating a functional configuration of inattentive state determination device 20C according to Embodiment 4.

As shown by FIG. 11, inattentive state determination device 20C includes user information obtainer 21, environmental information obtainer 22, drowsiness predictor 23, and inattentiveness determiner 25. In inattentive state determination device 20C, inattentiveness likelihood determiner 27 is disposed in inattentiveness determiner 25.

Inattentiveness likelihood determiner 27 determines a likelihood of an inattentive state, based on information obtained by user information obtainer 21 and information obtained by environmental information obtainer 22. Specifically, inattentiveness likelihood determiner 27 determines whether user U is likely to be in the inattentive state or an environment surrounding user U is likely to induce user U to be in the inattentive state.

For example, inattentiveness likelihood determiner 27 determines a likelihood of inattentiveness, based on a schedule or a conversation log (conversation record) of user U. For example, it is conceivable that user U is likely to be in the inattentive state before a tense meeting or before or after a quarrel with anther person or a brainstorming in an office. Moreover, inattentiveness likelihood determiner 27 may determine the complexity of a conversation depending on who a conversation partner is, and determine whether user U is likely to be in the inattentive state, in consideration of a psychological burden on user U.

Furthermore, when user U drives vehicle 91, a likelihood of the inattentiveness of user U may be determined based on, for example, the user U′s familiarity with driving, the vehicle model, a route, the user U′s driving experience, or the user U′s age. For example, it may be determined that user U drives awkwardly when driving vehicle 91 or taking a route for the first time, but user U is unlikely to be in the inattentive state. For example, a newly licensed driver drives roughly because such a driver is tense, but since the driver is not in the inattentive state, an inattentiveness likelihood determination line may be adjusted for the driver. For example, a likelihood of inattentiveness may be determined in consideration of the maintenance status of a road. For example, since user U tends to shift a gaze less when driving on a highway, an inattentiveness likelihood determination line may be adjusted according to the tendency.

Inattentiveness determination 25 may correct a degree of inattentiveness, based on the likelihood of the inattentive state determined by inattentiveness likelihood determiner 27, and may determine whether the person is in the inattentive state. When inattentiveness determiner 25 may correct a degree of inattentiveness by adding a weight to the degree of inattentiveness. Moreover, inattentiveness determiner 25 may correct not a degree of inattentiveness but a degree of drowsiness by adding a weight to the degree of drowsiness. Furthermore, inattentiveness determiner 25 may learn about past occurrences of inattentive states, based on a result of the determination by inattentiveness likelihood determiner 27, and may change a weight for a degree of inattentiveness when a current occurrence of an inattentive state is similar to the past occurrences.

In this way, inattentiveness determiner 25 according to Embodiment 4 determines a likelihood of an inattentive state, based on information obtained by user information obtainer 21 and information obtained by environmental information obtainer 22, and further determines whether a person is in the inattentive state, based on the likelihood of the inattentive state.

As stated above, determining a likelihood of an inattentive state makes it possible to distinguish between the inattentive state and the drowsiness occurrence state accurately. Accordingly, it is possible to determine accurately whether a person is in the inattentive state. It should be noted that the configuration of Embodiment 4 is applicable to any one of Embodiments 1 to 3.

Aforementioned Embodiments 1 to 3 each have described the relationship between the inattentive state and the drowsiness occurrence state, but the present disclosure is not limited to these examples. For example, since there is a correlation between the occurrence of drowsiness and the occurrence of fatigue, the same holds true for the inattentive state and a fatigue occurrence state. In view of this, Embodiments 5 to 7 each describe an inattentive state determination device with a focus on a relationship between the inattentive state and the fatigue occurrence state.

### Embodiment 5

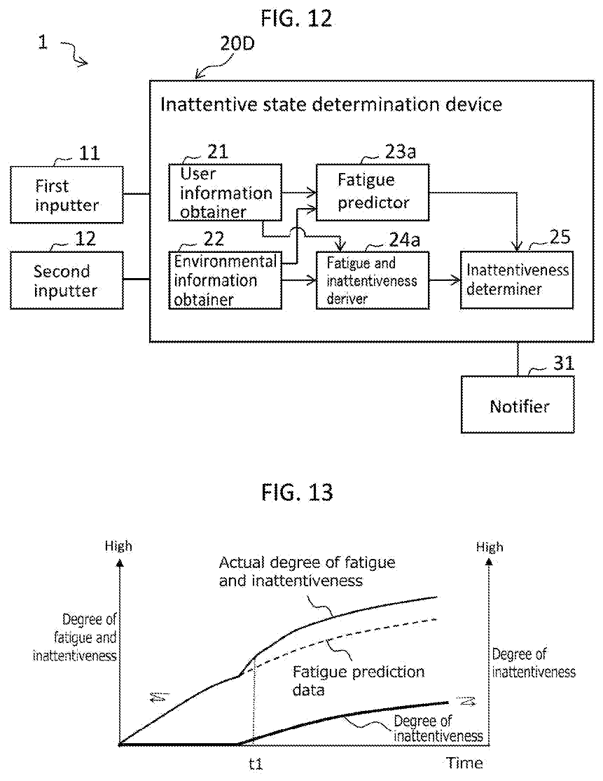

Next, the following describes a configuration of inattentive state determination device 20D according to Embodiment 5 with reference to FIG. 12 and FIG. 13. Inattentive state determination device 20D according to Embodiment 5 differs from Embodiment 1 in including fatigue predictor 23a and fatigue and inattentiveness deriver 24a.

FIG. 12 is a block diagram illustrating a functional configuration of inattentive state determination device 20D.

As shown by FIG. 12, inattentive state determination device 20D includes user information obtainer 21, environmental information obtainer 22, fatigue predictor 23a, fatigue and inattentiveness deriver 24a, and inattentiveness determiner 25.

User information obtainer 21 obtains information about user U inputted by first inputter 11 and second inputter 12. The information obtained by user information obtainer 21 is outputted to fatigue predictor 23a and fatigue and inattentiveness deriver 24a.

Environmental information obtainer 22 obtains information about an environment inputted by first inputter 11 and second inputter 12. The information obtained by environmental information obtainer 22 is outputted to fatigue predictor 23a and fatigue and inattentiveness deriver 24a.

Fatigue predictor 23a predicts the occurrence of fatigue in a person. Specifically, fatigue predictor 23a derives fatigue prediction data for predicting the occurrence of fatigue in user U, based on the information about user U obtained by user information obtainer 21 and the information about the environment obtained by environmental information obtainer 22.

Fatigue prediction data is a function for predicting the future occurrence of fatigue in user U. Fatigue prediction data can be derived by, for example, conducting multivariate analysis on data about degrees of fatigue in user U for past 10 minutes when user U was not in an inattentive state. It should be noted that a degree of fatigue is a value indicating a progress of fatigue. The degree of fatigue is represented by, for example, a five-point scale, and the value increases with an increase in the degree of fatigue. For example, a degree of fatigue is 1 when a person's blinking is periodically stable, 3 when the person's blinking is slow, and 5 when the person's eyes are closed.

Moreover, fatigue predictor 23a predicts the occurrence of fatigue, based on information about an action of user U such as working hours, a working posture, or an activity log (activity record). Specifically, fatigue predictor 23a predicts that fatigue is more likely to occur when working hours are longer, when the same operation or posture is repeated more for a long time, or more shortly after an exercise is finished. The fatigue prediction data obtained by fatigue predictor 23a is outputted to inattentiveness determiner 25.

Fatigue and inattentiveness deriver 24a derives a degree of fatigue and inattentiveness indicating a degree of fatigue and a degree of inattentiveness of a person. Specifically, fatigue and inattentiveness deriver 24a derives a current degree of fatigue and inattentiveness of user U, based on the information about user U obtained by user information obtainer 21 and the information about the environment obtained by environmental information obtainer 22.

It is difficult to determine whether a person is in a fatigue occurrence state or an inattentive state by merely checking the appearance of the person. For this reason, a degree of fatigue and inattentiveness obtained based on information about user U and information about an environment is a value including both a degree of fatigue and a degree of inattentiveness. The degree of fatigue and inattentiveness is represented by, for example, a ten-point scale, and the value increases with an increase in the degree of fatigue and inattentiveness. For example, a degree of fatigue and inattentiveness is 1 when a gaze of a person is frequently shifted, 5 when the gaze is shifted slowly, and 10 when the that gaze is completely fixed.

Fatigue and inattentiveness deriver 24a derives a degree of fatigue and inattentiveness, based on, for example, a grip strength, a response time, a posture, or breath components. Specifically, fatigue and inattentiveness deriver 24a determines a degree of fatigue and inattentiveness to be high when a grip strength is reduced, a response time is delayed, a posture is worsened, or the CO₂ concentration of breath is high. The degree of fatigue and inattentiveness derived by fatigue and inattentiveness deriver 24a is outputted to inattentiveness determiner 25.

Inattentiveness determiner 25 determines whether a person is in an inattentive state. Specifically, inattentiveness determiner 25 derives a degree of inattentiveness of a person, based on fatigue prediction data derived by fatigue predictor 23a and a degree of fatigue and inattentiveness obtained by fatigue and inattentiveness deriver 24a, and determines whether the person is in the inattentive state. The following describes how inattentiveness determiner 25 determines an inattentive state with reference to FIG. 13.

FIG. 13 is a diagram illustrating an example of determining an inattentive state using inattentive state determination device 20D.

The horizontal axis, left vertical axis, and right vertical axis of FIG. 13 indicate a time for monitoring user U, a degree of fatigue and inattentiveness, and a degree of inattentiveness, respectively. In FIG. 13, an actual degree of fatigue and inattentiveness obtained by fatigue and inattentiveness deriver 24a is shown by the solid line, and fatigue prediction data derived by fatigue predictor 23a is shown by the broken line.

As shown by FIG. 13, since fatigue gradually progresses with time, fatigue prediction data is shown by a gentle upward curve. In contrast, an inattentive state occurs depending on user U and appears as a sudden change in the middle of monitoring user U. For this reason, when an inattentive state occurs, a curve showing an actual degree of fatigue and inattentiveness considerably deviates from a curve showing fatigue prediction data.

Using these characteristics, it is possible to represent a degree of inattentiveness with a difference (see the thick solid line in FIG. 13) between an actual degree of fatigue and inattentiveness and fatigue prediction data. For example, the degree of inattentiveness at time t1 is a value obtained by subtracting a value of the fatigue prediction data at time t1 from a value of the actual degree of fatigue and inattentiveness at time t1. A predetermined threshold value may be set for a degree of inattentiveness in order to determine whether user U is in the inattentive state. When the degree of inattentiveness derived above is greater than or equal to the predetermined threshold value, user U may be determined to be in the inattentive state; and when the degree of inattentiveness derived above is less than the predetermined threshold value, user U may be determined to be not in the inattentive state. In this manner, it is possible to determine whether user U is in the inattentive state at time t1.

It should be noted that in the case where a degree of fatigue and inattentiveness and a degree of inattentiveness have a mutually different evaluation scale, it is desirable to, when a degree of inattentiveness is derived, make the evaluation scales conform to one another by performing predetermined weighting on a difference between an actual degree of fatigue and inattentiveness and fatigue prediction data.

Inattentive state determination device 20D according to the present embodiment includes: fatigue predictor 23a that derives fatigue prediction data for predicting the occurrence of fatigue in a person; fatigue and inattentiveness deriver 24a that derives a degree of fatigue and inattentiveness of the person; and inattentiveness determiner 25 that determines whether the person is in an inattentive state, based on the fatigue prediction data and a current degree of fatigue and inattentiveness.

As stated above, determining an inattentive state based on fatigue prediction data and a degree of fatigue and inattentiveness makes it possible to distinguish between the inattentive state and the fatigue occurrence state. Accordingly, it is possible to determine accurately whether a person is in the inattentive state.

### Embodiment 6

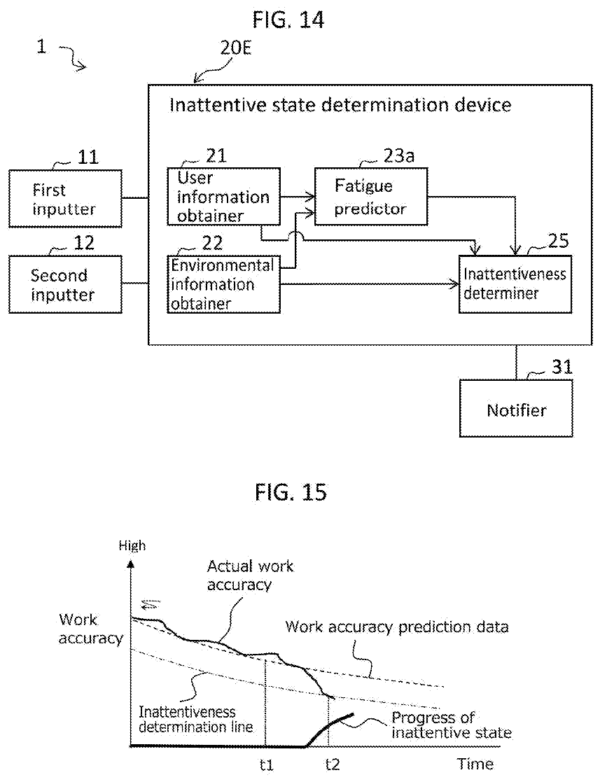

Next, the following describes inattentive state determination device 20E according to Embodiment 6 with reference to FIG. 14 and FIG. 15. Embodiment 6 describes an example of determining an inattentive state using prediction data obtained by fatigue predictor 23a but not using a degree of fatigue and inattentiveness.

FIG. 14 is a block diagram illustrating a functional configuration of inattentive state determination device 20E according to Embodiment 6. FIG. 15 is a diagram illustrating an example of determining an inattentive state using inattentive state determination device 20E.

As shown by FIG. 14, inattentive state determination device 20E includes user information obtainer 21, environmental information obtainer 22, fatigue predictor 23a, and inattentiveness determiner 25. Inattentive state determination device 20E according to Embodiment 6 does not include fatigue and inattentiveness deriver 24a.

Fatigue predictor 23a predicts the occurrence of fatigue in a person. Specifically, fatigue predictor 23a derives work accuracy prediction data (see FIG. 15) related to fatigue prediction data, based on information about user U obtained by user information obtainer 21 and information about an environment obtained by environmental information obtainer 22. Work accuracy refers to the accuracy of work finished in a predetermined period, and a lower work accuracy indicates a higher degree of fatigue.

Work accuracy prediction data is a function for predicting a future work accuracy of user U. Work accuracy prediction data is derived based on, for example, the above-described fatigue prediction data. Moreover, fatigue predictor 23a derives an inattentiveness determination line (see FIG. 15) that is a threshold value for determining whether a person is in an inattentive state. An inattentiveness determination line is provided along work accuracy prediction data and at predetermined intervals with respect to the work accuracy prediction data. It should be noted that the inattentiveness determination line may be provided such that the predetermined intervals vary according to a value of the work accuracy prediction data. The work accuracy prediction data obtained by fatigue predictor 23a is outputted to inattentiveness determiner 25.

It should be noted that the work accuracy prediction data has been shown as data related to fatigue prediction data in the above, the present embodiment is not limited to this example. For example, data related to fatigue prediction data may be driving accuracy prediction data.

Inattentiveness determiner 25 determines whether a person is in an inattentive state. Specifically, inattentiveness determiner 25 determines whether the person is in the inattentive state, based on the work accuracy prediction data and the inattentiveness determination line derived by drowsiness predictor 23a. The following describes how inattentiveness determiner 25 determines an inattentive state with reference to FIG. 15.

The horizontal axis and left vertical axis of FIG. 15 indicate a time for monitoring user U and a work accuracy, respectively. In FIG. 15, an actual work accuracy obtained by user information obtainer 21 is shown by the solid line, and work accuracy prediction data derived by fatigue predictor 23a is shown by the broken line.

As shown by FIG. 15, since fatigue gradually progresses with time, work accuracy prediction data is shown by a gentle downward curve. In contrast, an inattentive state occurs depending on user U and appears as a sudden change in the middle of monitoring user U. For this reason, when an inattentive state occurs, a curve showing an actual work accuracy considerably deviates from a curve showing the work accuracy prediction data.

FIG. 15 shows an example in which an inattentive state does not occur at time t1 but occurs at time t2. As shown by FIG. 15, in the present embodiment, when an actual work accuracy considerably deviates from prediction data and crosses an inattentiveness determination line, an inattentive state is determined to have occurred.

Inattentive state determination device 20E according to Embodiment 6 includes: fatigue predictor 23a that derives fatigue prediction data for predicting the occurrence of fatigue in a person; and inattentiveness determiner 25 that determines whether the person is in an inattentive state, based on the fatigue prediction data.

As stated above, determining an inattentive state based on fatigue prediction data makes it possible to distinguish between the inattentive state and the fatigue occurrence state. Accordingly, it is possible to determine accurately whether a person is in the inattentive state.

### Embodiment 7

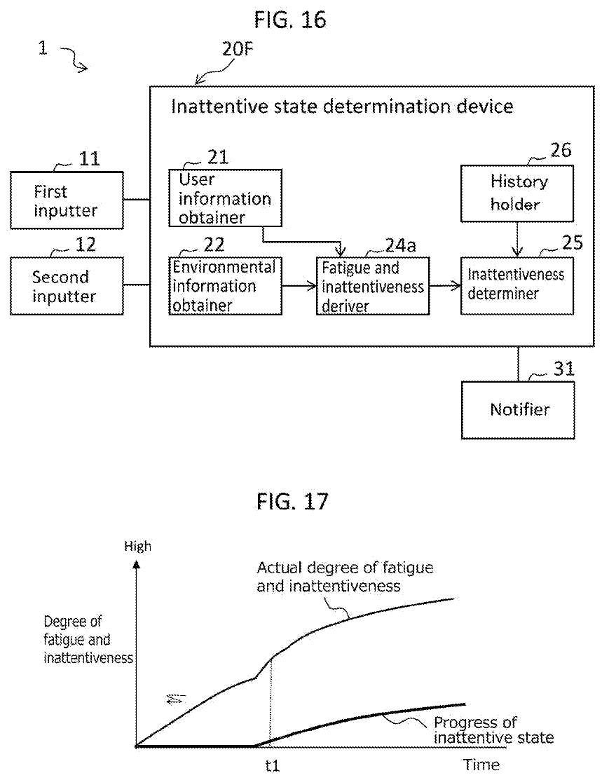

Next, the following describes inattentive state determination device 20F according to Embodiment 7 with reference to FIG. 16 and FIG. 17. Embodiment 7 describes an example of determining an inattentive state, based on a change in an actual degree of fatigue and inattentiveness, without using fatigue predictor 23a.

FIG. 16 is a block diagram illustrating a functional configuration of inattentive state determination device 20F according to Embodiment 7. FIG. 17 is a diagram illustrating an example of determining an inattentive state using inattentive state determination device 20F.

As shown by FIG. 16, inattentive state determination device 20F includes user information obtainer 21, environmental information obtainer 22, fatigue and inattentiveness deriver 24a, and inattentiveness determiner 25. In addition, inattentive state determination device 20F includes history holder 26. Inattentive state determination device 20F according to Embodiment 7 does not include fatigue predictor 23a.

Fatigue and inattentiveness deriver 24a derives a degree of fatigue and inattentiveness indicating a degree of fatigue and a degree of inattentiveness of a person. Specifically, fatigue and inattentiveness deriver 24a derives a current degree of fatigue and inattentiveness of user U, based on information about user U obtained by user information obtainer 21 and information about an environment obtained by environmental information obtainer 22. The degree of fatigue and inattentiveness derived by fatigue and inattentiveness deriver 24a is outputted to inattentiveness determiner 25.

Inattentiveness determiner 25 determines whether a person is in an inattentive state. Specifically, inattentiveness determiner 25 determines whether the person is in the inattentive state, based on a change in the degree of fatigue and inattentiveness obtained by fatigue and inattentiveness deriver 24a. The following describes how inattentiveness determiner 25 determines an inattentive state with reference to FIG. 17.

The horizontal axis and left vertical axis of FIG. 17 indicate a time for monitoring user U and a degree of fatigue and inattentiveness, respectively. In FIG. 17, an actual degree of fatigue and inattentiveness obtained by fatigue and inattentiveness deriver 24a is shown by the solid line.

As shown by FIG. 17, since fatigue gradually progresses with time, the fatigue is shown by a gentle upward curve. In contrast, an inattentive state occurs depending on user U and appears as a sudden change in the middle of monitoring user U. For this reason, as shown by FIG. 17, when an inattentive state occurs, a curve showing a degree of fatigue and inattentiveness changes suddenly with respect to a curve showing a past degree of fatigue and inattentiveness. For example, differential values before and after a change in a degree of fatigue and inattentiveness may be compared in order to determine whether user U is in the inattentive state. When a difference between the differential values is greater than or equal to a predetermined value, user U may be determined to be in the inattentive state; and when the difference between the differential values is less than the predetermined value, user U may be determined to be not in the inattentive state. In this manner, it is possible to determine whether user U is in the inattentive state.

Inattentive state determination device 20F according to the present embodiment includes: fatigue and inattentiveness deriver 24a that derives a degree of fatigue and inattentiveness indicating a degree of fatigue and a degree of inattentiveness of a person; and inattentiveness determiner 25 that determines whether the person is in an inattentive state, based on a change in the degree of fatigue and inattentiveness.

As stated above, determining an inattentive state based on a change in a degree of fatigue and inattentiveness makes it possible to distinguish between the inattentive state and the fatigue occurrence state and to determine the inattentive state. Accordingly, it is possible to determine accurately whether a person is in the inattentive state.

It should be noted that inattentive state determination device 20F may include history holder 26 that holds a fatigue and inattentiveness history that is a history of degrees of fatigue and inattentiveness. In addition, inattentiveness determiner 25 may compare the fatigue and inattentiveness history and a current degree of fatigue and inattentiveness to determine whether the person is in the inattentive state. For example, history holder 26 may hold a fatigue and inattentiveness history for a past few minutes while updating the fatigue and inattentiveness history, and inattentiveness determiner 25 may determine that the person is in the inattentive state when a current degree of fatigue and inattentiveness changes with at least a predetermined amplitude with respect to the fatigue and inattentiveness history.

### Other Embodiments

Although the inattentive state determination devices according to one or more aspects of the present disclosure have been described based on the aforementioned embodiments, the present disclosure is not limited to the aforementioned embodiments. Various modifications to the aforementioned embodiments that can be conceived by a person with an ordinary skill in the art or forms obtained by combining constituent elements in the different embodiments, for as long as they do not depart from the essence of the present disclosure, may be included in the scope of one or more aspects of the present disclosure.

For example, although the aforementioned embodiments show an example in which each of a degree of drowsiness and a degree of fatigue is represented by a five-point scale, the present disclosure is not limited to this example, and each of a degree of drowsiness and a degree of fatigue may be represented by at least a six-point scale or at most a four-point scale. Moreover, although the aforementioned embodiments show an example in which each of a degree of drowsiness and inattentiveness a degree of fatigue and inattentiveness is represented by a ten-point scale, the present disclosure is not limited to this example, and each of a degree of drowsiness and inattentiveness a degree of fatigue and inattentiveness may be represented by at least an eleven-point scale or at most a nine-point scale.

### INDUSTRIAL APPLICABILITY

The present disclosure is useful as a device for determining whether a person is in an inattentive state while, for example, driving or working in a room.

1. An inattentive state determination device, comprising:
a drowsiness predictor that derives drowsiness prediction data for predicting an occurrence of drowsiness in a person; and
an inattentiveness determiner that determines whether the person is in an inattentive state, based on the drowsiness prediction data.

2. The inattentive state determination device according to claim 1, further comprising:
a drowsiness and inattentiveness deriver that derives a degree of drowsiness and inattentiveness indicating a degree of drowsiness and a degree of inattentiveness of the person, wherein the inattentiveness determiner determines whether the person is in the inattentive state, based on the drowsiness prediction data and a current degree of drowsiness and inattentiveness.

3. The inattentive state determination device according to claim 1, further comprising:
a user information obtainer that obtains information about the person; and
an environmental information obtainer that obtains information about an environment surrounding the person,
wherein the drowsiness predictor derives the drowsiness prediction data, based on the information obtained by the user information obtainer and the information obtained by the environmental information obtainer.

4. The inattentive state determination device according to claim 2, further comprising:
a user information obtainer that obtains information about the person; and
an environmental information obtainer that obtains information about an environment surrounding the person,
wherein the drowsiness and inattentiveness deriver derives the degree of drowsiness and inattentiveness, based on the information obtained by the user information obtainer and the information obtained by the environmental information obtainer.

5. The inattentive state determination device according to claim 2, further comprising:
a user information obtainer that obtains information about the person; and
an environmental information obtainer that obtains information about an environment surrounding the person,
wherein the inattentiveness determiner determines a likelihood of the inattentive state, based on the information obtained by the user information obtainer and the information obtained by the environmental information obtainer, and further determines whether the person is in the inattentive state, based on the likelihood of the inattentive state.

6. An inattentive state determination device, comprising:
a drowsiness and inattentiveness deriver that derives a degree of drowsiness and inattentiveness indicating a degree of drowsiness and a degree of inattentiveness of a person; and
an inattentiveness determiner that determines whether the person is in an inattentive state, based on a change in the degree of drowsiness and inattentiveness.

7. The inattentive state determination device according to claim 6, further comprising:
a history holder that holds a drowsiness and inattentiveness history that is a history of degrees of drowsiness and inattentiveness,
wherein the inattentiveness determiner compares the drowsiness and inattentiveness history and a current degree of drowsiness and inattentiveness to determine whether the person is in the inattentive state.

8. The inattentive state determination device according to claim 6, further comprising:
a user information obtainer that obtains information about the person; and
an environmental information obtainer that obtains information about an environment surrounding the person,
wherein the drowsiness and inattentiveness deriver derives the degree of drowsiness and inattentiveness, based on the information obtained by the user information obtainer and the information obtained by the environmental information obtainer.

9. The inattentive state determination device according to claim 8,
wherein the inattentiveness determiner determines a likelihood of the inattentive state, based on the information obtained by the user information obtainer and the information obtained by the environmental information obtainer, and further determines whether the person is in the inattentive state, based on the likelihood of the inattentive state.

10. An inattentive state determination device, comprising:
a fatigue predictor that derives fatigue prediction data for predicting an occurrence of fatigue in a person; and
an inattentiveness determiner that determines whether the person is in an inattentive state, based on the fatigue prediction data.

11. An inattentive state determination device, comprising:
a fatigue and inattentiveness deriver that derives a degree of fatigue and inattentiveness indicating a degree of fatigue and a degree of inattentiveness of a person; and
an inattentiveness determiner that determines whether the person is in an inattentive state, based on a change in the degree of fatigue and inattentiveness.
